(19) United States
(12) Patent Application Publication
(10) Pub. No.: US 2006/0292254 A1
(43) Pub. Date: Dec. 28, 2006

(54) Orally and nasally administered appetite suppressant

(76) Inventor: Robert J. More, Princeton, NJ (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/449,118

(22) Filed: Jun. 8, 2006

Related U.S. Application Data
(60) Provisional application No. 60/689,011, filed on Jun. 8, 2005.

(51) Int. Cl. A61K 36/81; A61K 31/24; A61K 31/16

(52) U.S. Cl. 424/760; 514/537; 514/630

(57) ABSTRACT

Methods are described for suppressing appetite through the nasal and oral administration of a vanillylamide, thereby desensitizing gustatory or olfactory nerves and reducing the appeal of food. Long term desensitization is particularly contemplated.

PRIORITY CLAIM

This application claims priority under 35 U.S.C. § 119(e) to U.S. Provisional Application Ser. No. 60/689,011, filed Jun. 8, 2005, entitled “Orally and Nasally Administered Appetite Suppressant,” which is incorporated herein by reference in its entirety.

### BACKGROUND OF THE INVENTION

1. Field of the Invention

The present invention is in the field of pharmaceutical compositions for the treatment of obesity and for affecting weight loss in individuals by inhibition of oral and olfactory neurosensory perception.

2. Description of the Related Art

Obesity is a disorder characterized by the accumulation of excess fat in the body. Obesity is emerging as a global problem and is a major factor for a number of co-morbidities such as coronary heart disease, hypertension, non-insulin dependent diabetes mellitus, pulmonary dysfunction, osteoarthritis and certain types of cancer.

Obesity has been defined in terms of body mass index (BMI). BMI is calculated as weight (kg)/[height(m)]2. In addition to those individuals who satisfy a strict definition of medical obesity, a significant portion of the adult population is overweight. These individuals would also benefit from the availability of an effective weight-loss composition. Current products to suppress appetite and control weight are generally drugs with undesirable side effects. The main factor causing the development of obesity is a positive energy balance through the decreased activity and increased energy intake. Weight loss and loss of body fat can thus be achieved by reducing food intake and/ or increasing energy expenditure.

Studies show that weight tends to decline after a certain age. The reason for the decline in weight with aging has been attributed to the normal decline in the taste and smell senses. The smell of food alone has been demonstrated to increase pancreatic polypeptide within the first 3 minutes and to increase colonic pressure. The sight and smell of food increase insulin secretion in the first 20 minutes and this rise in insulin is blocked by atropine, suggesting that the rise is vagally mediated. Patients with anorexia have been shown to have a diminished sense of smell and in case reports, a diminished sense of taste and smell have been associated with weight loss. The smell of food also increases appetite and food intake in restrained eaters.

Vanillylamides have long been known to have an effect on sensory perception. Capsaicin, for example, is a vanillylamide that has been utilized for centuries. The first written description of the sensory effects of capsaicin appeared in Christopher Columbus' journal on Jan. 15, 1493 (Weil, 1981). Columbus stated that the natives of the New World had a habit of incorporating red-hot chili peppers into almost every one of their meals. Capsaicin-containing peppers have been cultivated in South America since at least 5200 B.C. Since the introduction of capsicum plants to the Indies in the late sixteenth century, the consumption of capsaicin whether in the form of paprika or chili pepper has become worldwide. The uses of capsaicin in folk medicine range from hair restoration and appetite stimulation to the treatment of gastric ulcers and rheumatism. Capsaicin has been found to activate a sub-population of sensory neurons and to evoke a sensation of burning pain (Substance P/heat-related). Paradoxically, capsaicin can also have analgesic and anti-inflammatory actions on neurons (Oh et al, 1996). This observation has inspired attempts to develop novel analgesic drugs based on the selective action of capsaicin on neurons that respond to tissue damaging stimuli. Capsaicin has been used extensively as a cytotoxin, where application of high systemic doses of capsaicin damages the stimulated neurons either killing neurons in neonatal animals or destroying the peripheral axons in adult animals (Chard et al, 1995).

Capsaicin elicits sensation by binding to VR1 receptors. VR1 receptors are expressed in the endings of trigeminal nonciceptors that innervate the oral mucosa, afferent fibers of which project to the brainstem trigeminal complex. Electrophysiological and biochemical studies have shown that capsaicin excites nociceptors by increasing the permeability of the plasma membrane to cations (Oh et al, 1996). Capsaicin appears to work by depleting substance P and calcitonin-gene related peptide (CGRP) expressing neurons. Peripheral sensory neurons treated with capsaicin release Substance P, the neurotransmitter of choice in the pain sensory pathway, causing acute overstimulation and a sensation of burning pain (Schmid et al, 1998).

Capsaicin depolarizes and excites mammalian sensory neurons due to its effect on membrane potential yet the depolarization is not sustained. The initial depolarization to capsaicin is accompanied by a reduction in cell input resistance, which suggests that capsaicin acts to open ion channels (VR1-evoked) in the plasma membrane (Oh et al, 1996). The effect of capsaicin has been studied in sensory neurons in vivo, in vitro, and in culture and there is general agreement that capsaicin depolarizes mammalian nociceptors and decreases their input resistance in a concentration-dependent manner (Caterina et al, 1997). An apparent decline in depolarization in the continued presence of capsaicin is probably due partly to desensitization of the capsaicin receptors and calcium entry during depolarization, which subsequently activates calcium-activated potassium channels (Schmid et al, 1996).

It has been shown that Substance P present in the nerve terminals of primary afferent sensory neurons had been depleted by capsaicin or the possible capsaicin receptor/ion channel had been desensitized with progressive doses. (Schmid et al, 1998). After several stimulations, this is followed by depletion of these fibers and results in nerve destruction. In sensory neurons, a mixture of monovalent and divalent cations carries vanilloid-evoked currents. Current-voltage relations established for cells bathed in solutions of differing cationic compositions show that VR1 does not discriminate between monovalent cations yet does exhibit a notable preference for divalent cations and high permeability to Ca++. One consequence of the increased calcium uptake is a relatively long-lasting loss of voltage-activated calcium channel activity, which would be expected to uncouple electrical excitation of the neuron from neurotransmitter (substance P) release (Schmid et al, 1998). Indeed this effect probably explains that while capsaicin can cause release of neuropeptides from sensory neurons (presumably due to calcium entry through ligand-gated ion channels) it also inhibits electrically evoked release, an effect which may be responsible for its analgesic and anti-inflammatory effects (Kirchstein et al, 1997).

Electrophysiological analyses of vanilloid-evoked responses have shown them to be kinetically complex and to desensitize with continuous vanilloid exposure (Caterina et al, 1997). It is important to distinguish between two phenomena that have been included in the general heading of desensitization (Szolcsanyi, 1993). The first is a pharmacological desensitization where prolonged or repeated applications of capsaicin lead to a progressive decline in the size of subsequent responses to capsaicin. As mentioned above, the amount of Substance P in the nerve terminals had been depleted with escalating capsaicin treatment thus ‘desensitizing’ or reducing the effect of capsaicin in the pain pathway (Schmid et al, 1998). The second is a functional desensitization where a challenge with capsaicin leads to a reduction or loss of responsiveness to other stimuli. These two effects are referred to here as desensitization and functional desensitization. In multicellular preparations the extent of desensitization depends on the capsaicin concentration, how frequently the drug is applied and for how long. Functional desensitization depends, in part, on the presence of extracellular calcium. Indeed, a single application of capsaicin causes marked attenuation of response to subsequent challenges with the drug. Just as capsaicin-induced depolarization (inward current and increase in conductance) is dependent on the presence of a negative membrane potential, desensitization also seems to be a voltage-sensitive phenomenon. The voltage-dependence of desensitization at positive potentials probably arises, at least in part, due to the reduction in the electrochemical gradient for calcium, causing a decrease in calcium entry. A possible explanation for the phenomenon of desensitization presented by progressive intracellular increases in calcium concentration is the subsequent stimulation of calcineurin, a calcium and calmodulin-dependent cytosolic enzyme (Szolcsanyi, 1993). Tests have shown that in the presence of a complex of cyclophilin and cyclosporin A, specific calcineurin inhibitors, desensitization was almost completely eradicated. Intriguingly, the activation of calcineurin may be responsible for both pharmacological desensitization of VR1s and the long-term blocking of calcium conductances (discussed below in the neurotoxicity section). It is possible that functional desensitization of nociceptors is due to calcium-dependent de/phosphorylation of other substrates such as sodium ion channels or other regulatory enzymes. In accord with our definition of functional desensitization above, the presence of extracellular calcium is paramount for the stimulation of calcineurin and the subsequent functional desensitizaton of the vanilloid receptor.

### SUMMARY OF THE INVENTION

One embodiment of the invention is a method for reducing the appetite in a mammal including delivering to the mammal a nerve-desensitizing amount of a vanillylamide so as to effect long-term desensitization of taste-related nerves which is effective in reducing appetite.

In a preferred embodiment, the vanillylamide can be capsaicin.

In another preferred embodiment, the vanillylamide can be applied topically to the tongue.

In another preferred embodiment, the vanillylamide may be applied subdermally to the oral mucosa.

In some embodiments, the vanillylamide is selected from capsaicin, dihydrocapsaicin, nordihydrocapsaicin, oleoresin capsicum and civamide.

Other embodiments also include application of an analgesic to the area to which the vanillylamide is applied.

In a preferred embodiment, the analgesic is lidocaine.

In some embodiments, the vanillylamide can be injected into the tongue following administration of an anesthetic to the tongue.

In a preferred embodiment, the amount of vanillylamide can be between about 1 mg and about 1000 mg.

In another embodiment, the method includes instilling capsaicin into the nasal passageways to effect contact with olfactory nerves.

In some embodiments, the instilling step can be repeated at least two times within about one month.

Another embodiment of the invention is a composition for reducing appetite in a mammal, including a vanillylamide in a fast-melt delivery form.

In a preferred embodiment, the concentration of the vanillylamide is between about 1 ppm and about 1000 ppm.

In another preferred embodiment, the vanillylamide is capsaicin.

Another embodiment of the invention is a method of treating obesity in a mammal in need of such treatment including the steps of injecting an analgesic into the oral mucosa of the mammal and injecting a vanillylamide into the oral mucosa of the mammal.

In a preferred embodiment, the analgesic can be lidocaine.

In another preferred embodiment, the vanillylamide is capsaicin.

Another embodiment of the invention is a method of treating obesity in a mammal in need of such treatment including the steps of applying a decongestant to the inside of a nostril of the mammal, applying an anesthetic to the inside of the nostril and applying a vanillylamide to the inside of the nostril.

In one preferred embodiment, the anesthetic is a topical anesthetic.

In some embodiments, the anesthetic is selected from lidocaine, prilocaine, benzocaine, tetracaine, phenylephrine and pramoxine.

In a preferred embodiment, the vanillylamide is capsaicin.

### DETAILED DESCRIPTION OF THE INVENTION

The effect that the smell and taste of food has on the body and appetite is well documented. Therefore, temporarily depressing the sense of taste and smell prior to meals may be an effective strategy to decrease food intake. Embodiments of the invention relate to methods of appetite suppression through the inhibition of oral and olfactory neurosensory perception. In one embodiment, a vanillylamide is applied intranasally to a mammal in need of weight loss. Capsaicin, for example can be applied to the nostrils of a subject by way of an aerosol spray or nose drops to desensitize the tissue and reduce sensory perception. With a reduced sensory perception of the food in the meal, the subject exhibits a reduced physiological response which results in lower food intake.

In another embodiment of the invention, a vanillylamide is applied to the oral mucosa of a subject, either topically or subdermally. The tongue has a rich sensory innervation and serves both general (concerning pain, temperature, touch, etc.) and special (concerning taste) sensory functions. These two distinct classes of sensory information are conducted to the central nervous system by distinctive nerve bundles. By interrupting the pathway of sensation, the vanillylamide can be used to lower appetite and ultimately, food intake in a subject.

One embodiment of the present invention involves a long term desensitization of the oral or olfactory nerves. Thus, unlike short-term effects that may occur upon eating capsaicin-containing foods, one aspect of the present invention focuses on achieving a longer-term desensitization. This longer term desensitization preferably extends for at least 2, 3, or 5 days, preferably at least about 1, 2, or 4 weeks, and more preferably the desensitization lasts 1 or 2 months, or longer.

In order to achieve this long-term effect, the amount of vanillylamide and the contact time with the target tissue are carefully selected. Thus, for example, direct injection into the tongue of a capsaicin solution containing about 1 mg to about 1000 mg and 1, 2, 4, 10, or more injections into the tongue. For intranasal use, topical treatment is preferred. The form of administration can be, for example, via drops, spray, or instillation of a bolus liquid dose. Due to reduced tissue penetration through such topical administration, the dosage is typically repeated at regular intervals, e.g., every day or two, or even before every meal. However, a series of topical intranasal dosages can provide longer term desensitization, e.g., when repeated every other day for two weeks.

In one preferred embodiment, analgesics or anesthetics can be used as a pretreatment or cotreatment to decrease the burning sensation associated with vanillylamides. Preferably, the analgesic or anesthetic is administered prior to administration of the vanillylamide. Although dosages necessary to achieve localized anesthesia will vary depending on the anesthetic used, such anesthetics and their dosages are well known in the art, and conventional dosages can be utilized in the present invention. Preferably, the anesthetic is administered sufficiently ahead of the vanillylamide treatment to permit analgesia to take effect prior to administration of the vanillylamide. Any commercially available analgesics or anesthetics can be used with the invention. Some examples of those that can be used include lidocaine, benzocaine, xylocaine, dibucaine, tetracain, pramoxine and bupivacaine. These materials can be applied topically or by injection.

In addition to the long term desensitization treatments contemplated in the present invention, short term nasal treatment is also contemplated, in which the amount of capsaicin or other vanillylamide is sufficient to evoke only a short term response; e.g., an individual dosage of from about 1 ppm to about 1000 ppm of capsaicin in an appropriate vehicle. In addition to drops or sprays for nasal administration, a fast melt composition is particularly contemplated for oral use. Fast melt strips, for example, are well-known dosage forms for pharmaceutical or cosmetic uses, and are typically provided as a rapidly-dissolving strip for placement on the tongue, which provides an almost instant oral dosage of the desired material. Here, strips containing capsaicin alone, or with a flavoring ingredient, or with a topical anesthetic are specifically contemplated.

Although capsaicin is a preferred vanillylamide, other vanillylamides having similar nerve-disabling effects are also contemplated including dihydrocapsaicin ordihydrocapsaicin, oleoresin capsicum, civamide and other VR1 agonists.

Vanillylamides are preferred over other currently available weight loss drugs because of reduced side effects. Many weight loss drugs currently sold have many negative side effects which range from merely unpleasant to hazardous. Vanillylamides, on the other hand, have very few side effects. Capsaicin, for example, has been a common food ingredient for thousands of years.

Certain aspects of the present invention are further exemplified as follows:

### EXAMPLE 1

### Effect of Intranasal Administration of Capsaicin on Food Intake in Food Deprived Rats

This Example shows that Capsaicin administration results in a reduced food intake in an animal model. Male Sprague-Dawley rats, 150-175 g at the start of the experiments (Charles River Laboratories, Wilmington, Mass.) were housed individually in suspended wire mesh cages. Rats had ad libitum access to tap water and food (Rodent Laboratory Chow No. 5001-pellet form, Purina) on non-testing days. A mush diet, used for experiments on food-deprived animals, and composed of equal parts by weight of ground rodent chow (Purina, #5001-meal form) and a 4% nutrient agar solution (Teklad Diets) was presented to the animals in glass jars. This agar-based chow diet allows for a more accurate measurement of food intake, and has been shown previously to be sufficient for maintaining normal rat growth.

Capsaicin was prepared as a liquid solution and administered intra-nasally, The drug dosage ranged from 0.02-2 mg/kg of body weight. Control animals were administered placebo formulation without capsaicin. One hour after administration, pre-weighed food jars were introduced into the cage and a sheet of clean white paper placed under each cage to catch any spillage. The amount of food consumed after 4, 12 and 24 hours was determined to the nearest 0.01 g. Rats given the drug consumed significantly less food than rats on placebo.

### EXAMPLE 2

Thirty healthy non-dieting subjects 18 years of age of older with BMI between 30-40 and on no regular medication participate in a trial. Three applications of xylometazolinehydrochloride 0.1% (Otrivin R 1 mg/mL; Zyma, Breda, Holland; nebulizator) were given for decongestion in each nostril. The nasal airway was anaesthetized by three applications (10 mg/puff) of lidocaine base (100 mg/mL; Xylocaine R 10% spray; Astra, Rijswijk, Holland) in each nostril. To ensure good anaesthesia, a pause of 15 min was introduced. Lips,columella and philtrum were covered with a petrolatum/lanolin/glycerin salve. Patients were instructed to inhale deeply before, hold their breath during, and exhale after substance application. The capsaicin solution (0.1 mmol/L) consisted of pelargonic acid vanillylamide (Fluka, Buchs,Germany) dissolved in 3 mL alcohol (96%) and diluted in 1 L NaCI solution (0.9%) During provocation, 0.5 mL solution was sprayed into each nostril (0.15 mg capsaicin). In 14 days, seven applications of capsaicin or placebo were given at 2-day intervals. At meal-time, the subjects were instructed to eat ad libitum in the laboratory until satiated. To facilitate the measurement of food intake, food was prepared and pre-portioned. After each meal, quantities of foods that were not entirely consumed were reweighed to determine net intake of each food. Prospective food consumption, desire to eat, hunger, fullness and satiety were measured immediately before and after the breakfast and lunch by using a 150 mm visual analogue scale.

### EXAMPLE 3

Thirty healthy non-dieting subjects 18 years of age of older with BMI between 30-40 and on no regular medication participate in a trial. The oral mucusa in these patients were anaesthetized by injection of lidocaine base (100 mg/mL Xylocaine). The oral irritant capsaicin (Sigma, St. Louis) was used at a concentration of 109 uM. Capsaicin was initially dissolved as a stock solution of 3.3 mM in a solution containing 50% ethanol. Working capsaicin concentrations contained 1.65% ethanol. Intraarterial (i.a.) injections of capsaicin were performed. On days 7 and 14, participants come to the laboratory to eat breakfast, lunch, and dinner so that daily energy and macronutrient intakes and ratings of hunger and satiety can be measured.

### EQUIVALENTS

The foregoing written specification is considered to be sufficient to enable one skilled in the art to practice the invention. The foregoing description details certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A method for reducing the appetite in a mammal comprising delivering to the mammal a nerve-desensitizing amount of a vanillylamide so as to effect long-term desensitization of taste-related nerves which is effective in reducing appetite.

2. The method of claim 1, wherein the vanillylamide is capsaicin.

3. The method of claim 1, wherein the vanillylamide is applied topically to the tongue.

4. The method of claim 1, wherein the vanillylamide is applied subdermally to the oral mucosa.

5. The method of claim 1, wherein the vanillylamide is selected from capsaicin, dihydrocapsaicin, nordihydrocapsaicin, oleoresin capsicum and civamide.

6. The method of claim 1, further comprising application of an analgesic to the area to which the vanillylamide is applied.

7. The method of claim 6, wherein the analgesic is lidocaine.

8. The method of claim 1, wherein the vanillylamide is injected into the tongue following administration of an anesthetic to the tongue.

9. The method of claim 1, wherein the amount of vanillylamide is between about 1 mg and about 1000 mg.

10. The method of claim 1, comprising instilling capsaicin into the nasal passageways to affect contact with olfactory nerves.

11. The method of claim 10, wherein the instilling step is repeated at least two times within about one month.

12. A composition for reducing appetite in a mammal, comprising a vanillylamide in a fast-melt delivery form.

13. The composition of claim 12, wherein the concentration of the vanillylamide is between about 1 ppm and about 1000 ppm.

14. The composition of claim 12, wherein the vanillylamide is capsaicin.

15. A method of treating obesity in a mammal in need of such treatment comprising:
injecting an analgesic into the oral mucosa of the mammal; and
injecting a vanillylamide into the oral mucosa of the mammal.

16. The method of claim 15, wherein the analgesic is lidocaine.

17. The method of claim 15, wherein the vanillylamide is capsaicin.

18. A method of treating obesity in a mammal in need of such treatment comprising:
applying a decongestant to the inside of a nostril of the mammal;
applying an anesthetic to the inside of the nostril; and
applying a vanillylamide to the inside of the nostril.

19. The method of claim 18, wherein the anesthetic is a topical anesthetic.

20. The method of claim 18, wherein the anesthetic is selected from the group consisting of: lidocaine, prilocaine, benzocaine, tetracaine, phenylephrine and pramoxine.

21. The method of claim 18, wherein the vanillylamide is capsaicin.
